(19) United States
(12) Patent Application Publication
(10) Pub. No.: US 2008/0194698 A1
(43) Pub. Date: Aug. 14, 2008

(54) Nmda Receptor Antagonists in the Medical Intervention of Metabolic Disorders

(76) Inventors: Michael Hermanussen, Altenhof (DE); Jesus Angel Fernandez-Tresguerres, Madrid (ES)

Correspondence Address: MUETING, RAASCH & GEBHARDT, P.A., P.O. BOX 581336, MINNEAPOLIS, MN, 55458-1336, US

(21) Appl. No.: 11/885,696

(22) PCT Filed: Feb. 27, 2006
(86) PCT No.: PCT/EP06/01802

§ 371 Date: Mar. 4, 2008

Related U.S. Application Data
(60) Provisional application No. 60/661,127, filed on Mar. 11, 2005.
(60) Provisional application No. 60/673,303, filed on Apr. 20, 2005.

(30) Foreign Application Priority Data
Mar. 7, 2005 (EP) 05004957.6

(51) Int. Cl. A61K 31/13; A61P 3/00; A61P 3/04

(52) U.S. Cl. 514/662; 514/579

(57) ABSTRACT

The present invention provides for the use of an NMDA receptor antagonist, preferably memantine or neramexane or a pharmaceutical acceptable salt or a prodrug of said antagonists, in the preparation of a pharmaceutical composition for the prevention, amelioration and/or treatment of disorders of metabolism influencing body weight, in particular obesity, an eating disorder and/or in the regulation of appetite. Furthermore, the present invention provides for a method for the prevention, amelioration and/or treatment of disorders of metabolism influencing body weight, in particular obesity, an eating disorder and/or in the regulation of appetite comprising the step of administering to a subject in need thereof a therapeutically effective amount of an NMDA receptor antagonist, preferably memantine or neramexane or a pharmaceutically acceptable salt or prodrug of said antagonists.

The present invention provides for the use of an NMDA receptor antagonist, in particular of memantine or neramexane or a pharmaceutically acceptable salt or a prodrug of said NMDA receptor antagonists in the preparation of a pharmaceutical composition for the prevention, amelioration and/or treatment of disorders of metabolism influencing body weight, in particular obesity, an eating disorder and/or in the regulation of appetite. Furthermore, the present invention provides for a method for the prevention, amelioration and/or treatment of disorders of metabolism influencing body weight, in particular obesity, an eating disorder and/or in the regulation of appetite comprising the step of administering to a subject in need thereof a therapeutically effective amount of an NMDA receptor antagonist, in particular of memantine or neramexane or a pharmaceutically acceptable salt or a prodrug of said NMDA receptor antagonists.

World-wide obesity has risen to alarming levels (McLellan, Lancet (2002), 359:1412). The average weight of German conscripts now increases by almost 400 g/year. Similar data were obtained in Austria, Norway and the UK. Obesity is not a separate problem of only the obese people but appears to be a characteristic feature of modern populations as a whole (Hermanussen, Int. J. Obesity (2001), 25:1550-3).

Obesity is a complex disorder of appetite regulation and/or energy metabolism controlled by specific biological factors. Besides severe risks of illness such as diabetes, hypertension and heart disease, individuals suffering from obesity are often isolated socially.

Human obesity is strongly influenced by environmental and genetic factors, whereby the environmental influence is often a hurdle for the identification of (human) obesity genes.

Obesity is defined as a Body Mass Index (BMI) of 30 kg/m² or more. BMI is calculated by dividing the weight in kg by the height in metres squared. “Overweight” is defined as a BMI between 25 and 30 kg/m². A person is considered obese if he or she has 20 percent (or more) extra body fat for his/her age, height, sex, and bone structure.

Obesity has a major impact on a person's physical, social and emotional well-being. Besides this, obesity can lead to an increased risk of illness including type 2 diabetes and high blood pressure (hypertension) that can lead to cardiovascular diseases. Obesity can also play a role in cancer, problems with sexual-function, muscle and bone disorders and dyslipidaemia.

Major advances have recently been made in identifying genetic components of the homeostatic system(s) that regulate body weight/mass. Several candidate genes have been associated with mammalian/human obesity or its metabolic complications (Kopelman, Nature 404 (2000), 634-643). One key element of the homeostatic system regulating body weight/mass is the hormone leptin (Friedman, Nature 395 (1998), 763-770; Friedman, Nature 404 (2000), 632-634; Chicurel, Nature 404 (2000), 538-540). Leptin is produced by fat tissue and reports nutritional information to key regulatory centers in the hypothalamus. A decrease in body fat leads to a decreased level of leptin, which in turn stimulates food intake. Furthermore, decreased leptin levels activate a hormonal response that is characteristic of a starvation state (Ahima, Nature 382 (1996), 250-252). Leptin acts on nerve cells in the brain and modulates this function. Several neuropeptides are implicated in the control of energy homeostasis, inter alia, neuropeptide Y (NPY) and agouti-related protein (AGRP), α-melanocyte-stimulating hormone (α-MSH) and cocaine—and amphetamine—regulated transcript (CART); see Friedman (2000), loc. cit.; Schwartz, Nature 404 (2000), 661-671; Erickson, Science 274 (1996), 1704-1707; Fan, Nature 385 (1997), 165-168.

The neuronal circuits furthermore regulate further effector molecules which have recently been identified (for review see Lowell, Nature 404 (2000), 652-660). These effector molecules comprise uncoupling proteins (UCP1, UCP2 and/or UCP3; Lowell (2000), loc. cit.) and peroxisome proliferator-activated receptors (PPAR-γ) co-activator (PGC-I), a key regulator of the genes that regulate thermogenesis (Puigserver, Cell 92 (1998), 829-839).

Furthermore, energy balance and thereby body weight/mass is modulated by the above mentioned neuropeptides and further (neurogenic) factors, like pro-opiomelanocortin (POMC), the precursor of α-MSH (Elias, Neuron 23 (1999), 775-786). Mutations in POMC are implicated in obesity (Krude, Nature Genetics 19 (1998), 155).

Additional mutations are described which cause modified and/or altered leptin responses. For example, in 3-5% of extreme obese individuals, mutations in the MSH receptor (MC4R), leading to leptin resistance, have been described (Friedman (2000), loc. cit.; Vaisse, Nature Gen. 20 (1998), 113-114). Mutations in the leptin receptor itself are also associated with extreme obesity (Clement, Nature 392 (1998), 398-401).

Accordingly, obesity is not to be considered as a single disorder but a heterogeneous group of conditions with (potential) multiple causes. Therefore, obesity is also characterized by elevated fasting plasma insulin and an exaggerated insulin response to oral glucose intake (Kolterman, J. Clin. Invest 65 (1980), 1272-1284) and a clear involvement of obesity in type 2 diabetes mellitus can be confirmed (Kopelman (2000), loc. cit.; Colditz, Arch. Int. Med. 122 (1995), 481-486).

As with other complex diseases, rare obesity mutations have been described which have been identified by mendelian pattern of inheritance and position mapping (see Barsh, Nature 404 (2000), 644-650). With one or two notable exceptions, the map positions of obesity loci identified by quantitative studies do not correspond to defined (mouse) obesity mutations such as ob (leptin), fat (carboxypeptidase E) or tubby (tubby protein). Map positions have been determined for some clinical syndromes, like Prader-Willi, Cohen, Alstrom, Bardet-Biedl or Borjeson-Forssman-Lehman, but the causative genes have not yet been isolated (see Barsh (2000), loc. cit.; Ohta, Am. J. Hum. Gen. 64 (1999), 397-413; Kolehmainen, Eur. J. Hum. Gen. 5 (1997), 206-213; Russell-Eggitt, Ophthalmology 105 (1998), 1274-1280; Mathews, Am. J. Med. Gen. 34 (1989), 470-474; Bruford, Genomics 41 (1997), 93-99). The “human obesity gene map” contains entries for more than 40 genes and 15 chromosomal regions in which published studies indicate a possible relationship to adiposity or a related phenotype (Barsh (2000), loc. cit., Perusse, Obes. Res. 7 (1999), 111-129). Said “obesity gene map” comprises, however, mainly large chromosomal areas and does not provide for distinct genes involved in obesity. Lately, Snyder (2003) has published an extended version of the “obesity gene map” and more than 430 genes, markers, chromosomal regions have been associated or linked with human obesity phenotypes (Snyder, Obes. Res. 12 (2004), 369-439).

Much effort has been spent to understand the pathophysiology of obesity. Apart from the rare monogenic causes for severe disturbances of the eating regulation—genetic alterations of the ob gene (leptin) (Zhang, Nature (1996), 372:425-32; Strobel, Nat. Tenet. (1998) 18:213-215), the leptin receptor (Clement, Nature (1998), 392:398-401), a mutation of the melanocortin 4 receptor (MC4R) gene (Farooqi, J. Clin. Invest. (2000), 106:271-279), and mutations in the pro-opiomelanocortin (POMC) gene (Krude, Nat. Genet. (1998), 19:155-157)—obesity appears to show a multifactorial etiopathogenesis. Disadvantageous dietary habits, such as over-consumption of fat-rich diets, excessive use of modern media, in particular television viewing (Robinson, Pediatr. Clin. N. Am. (2001), 48:1017-1025), a sedentary life style (Votruba, Nutrition (2000), 16:179-188), and many other exogenous factors, have been made responsible for the development of obesity already in early childhood. And recently, a new and very challenging hypothesis has been added linking obesity, voracity and growth hormone deficiency, to the consumption of elevated amounts of the amino acid glutamate (GLU) (Hermanussen, Tresguerres, J. Pediatr. Endocrinol. Metabol. (2003), 16:965-968; Hermanussen, Tresguerres J. Perinat. Med. (2003), 31:489-495; Hermanussen M, García A P, Sunder M, Voigt M, Salazar V, Tresguerres JAF Eur J Clin Nutr (2005) (advance online publication 30.8.2005)).

The arcuate nucleus is the major site of GLU-induced neuronal damage in the hypothalamus. It is situated close to the bottom of the third ventricle, and is a potent site of leptin action. Leptin is produced in the adipose tissue, crosses the blood-brain barrier by active transport systems, and stimulates a specific signalling cascade (Jequier, Ann. NY Acad. Sci. (2002), 967: 397-388): It downregulates the orexigenic neuropeptides NPY, agouti gene-related protein, melanin-concentrating hormone, and orexins, and upregulates pro-opiomelanocortin (POMC) and cocaine- and amphetamine-regulated transcript (CART) mRNA (Elmquist, Int. J. Obes. Relat. Metab. Disord. (2001) 25:S78-82). POMC and its post-translational product, alpha-MSH, stimulate melanocortin receptors (MC3R, MC4R) and thereby down-regulate appetite. Arcuate nucleus damage disrupts the signalling cascade of leptin action, thereby impairs the regulation of appetite, and causes voracity (Lu, Psychopharmacol. Bull. (2001), 35:45-65; Fan, Nature (1997), 385:165-168).

GLU toxicity is mediated either by inhibiting cystine uptake (Murphy, FASEB J. (1990), 4:1624-1633) or receptor-mediated (excitotoxicity). Excitotoxicity is one of the most extensively studied processes of neuronal cell death, and plays an important role in many central nervous system (CNS) diseases, including CNS ischemia, trauma, and neurodegenerative disorders. Excitotoxicity is characterized as an excessive presence of glutamate, which in turn activates postsynaptic glutamate receptors. While almost every glutamate receptor subtype has been implicated in mediating excitotoxic cell death, it is generally accepted that the N-methyl-D-aspartate (NMDA) receptor subtypes play a major role, mainly owing to their high calcium (Ca2+) permeability (Sattler and Tymiansk, Mol. Neurobiol., August-December; 24 (1-3), (2001), 107-29).

The N-methyl-D-aspartate receptor (NMDA-R) is fully functional in the rat early in embryogenesis. Xue and co-workers (Xue, Acta Neuropathol. (Berl) (1997), 94:572-82) found that glutamate- and aspartate-immunoreactive neurones were completely absent in the monosodium glutamate (MSG)-lesioned arcuate nucleus as well as the ventromedial nucleus lateral to the arcuate nucleus, in mice treated neonatally with MSG. Similarly, NMDA-R1-immunoreactive neurones were mostly absent in the MSG-lesioned arcuate nucleus but remained intact in the ventromedial nucleus. There was also a substantial loss of NMDA-R2 immunoreactivity within the arcuate nucleus. Beas-Zarate and co-workers (Beas-Zarate, Neurochem. Int. (2001), 39:1-10) measured changes in gene expression of the NMDA-R subunits: NMDA-R1, NMDA-R 2A and NMDA-R 2B in cerebral cortex, striatum and hippocampus in the brains of rats treated neonatally with MSG. The authors showed increases in GLU levels and activation of GLU-receptors after neonatal s.c. administration of MSG at doses of 4 mg/g body weight and an increase in glial cell reactivity and important changes in NMDA-R molecular composition, with signs of neuronal damage. Kaufhold and co-workers (Kaufhold, J. Pharmacol. Exp. Ther. (2002), 302:490-6) were able to prevent the adverse effects of neonatal MSG treatment by concurrent administration of a selective and highly potent non-competitive N-methyl-D-aspartate receptor antagonist of GLU.

Administering GLU to newborn rodents not only destroys arcuate nucleus neurones, it also damages other hypothalamic areas. Bloch et al. (Bloch, Nature (1984), 307:272-3) showed that MSG treatment results in the complete loss of growth hormone releasing factor (GRF)-immunoreactive cell bodies within this nucleus and provokes a selective disappearance of GRF-immunoreactive fibres in the median eminence of rats. This technique has routinely been practised to produce functionally hypopituitary animals (Lima, Neuroendocrinology (1993), 57:1155-1166) for studies of short-term growth (Hermanussen, Growth Regulation (1996), 6:230-237).

GLU-induced neuronal damage results in voracity and subsequent excessive weight gain, as well as impaired growth hormone (GH) secretion, the two major characteristics of human obesity.

Known therapies for obese patients comprise in particular physical activity, diet as well as drug therapy.

Many drugs tested as an appetite suppressant interfere with monoamine-neurotransmitters (serotonin, noradrenalin, dopamine, histamine). 5-HT (5-hydroxytryptamine) is released in various sites of the hypothalamus, a brain region believed to be involved in the regulation of food intake. D-fenfluramine is a 5-HT releaser and reuptake inhibitor mostly used in combination with Phentermine (Fen-Phen) to treat obesity. Fen-Phen was withdrawn from the market due to potential heart valve defects (Wadden, Obes. Res. 7 (1999), 309-310). Recently sibutramine (Nakagawa et al. 2000), a novel 5-HT and noradrenalin reuptake inhibitor (Knoll Pharma; Bray, Obes. Res 7 (1999), 189-198) was shown to support weight loss when used to support a low calorie diet. Other drugs interfering with monoamine-neurotransmitter effects (e.g. drugs so far used as anti-depressants) are also discussed for their efficacy in the treatment of obesity (Sayler, Int. J. Obes. Realt. Metab. Disord. 18 (1994), 742-751; Wadden, Obes. Res. 3 (1995), 549-557). These drugs comprise topiramate and will be discussed below.

Orlistat (Xenical®) prevents the absorption of some fat in the intestine. Just under a third of the fat that would otherwise have been absorbed passes straight through the bowel and is excreted in the faeces.

Pregnenolone may have some efficacy as a memory enhancer. This has so far been demonstrated in various animal models but not yet in humans. There are unsubstantiated claims that pregnenolone is useful in Alzheimer's disease, some forms of cancer and arthritis, in degenerative diseases associated with aging in general and in obesity. Pregnenolone is available from numerous manufacturers generically. Branded products include MaxiLife Pregnenolone (Twinlab). Malayev et al. (2002) examined the effects of pregnenolone sulphate (PS) on various NMDA receptor subtypes. Whereas PS potentiated NMDA-, glutamate-, and glycine-induced currents of NR1/NR2A and NR1/NR2B receptors, it was inhibitory at NR1/NR2c and NR1/NR2D receptors. Horak et al. (2004) showed that responses of the NMDA receptors to glutamate recorded in the continuous presence of PS, exhibited a marked time-dependent decline. These and other observations indicate that the effectiveness of PS on the regulation of appetite is possibly mediated via the NMDA receptor.

Pregnenolone is a steroid naturally found in animal tissues, especially in the gonads, adrenal gland and brain. Pregnenolone is synthesized from cholesterol and is a precursor for the biosynthesis of steroid hormones. In the adrenal gland, pregnenolone is a precursor to the mineralocorticoid aldosterone, the glucocorticoid cortisol, as well as dehydroepiandrosterone (DHEA) and progesterone. In the ovary, pregnenolone is a precursor to estrogens and progesterone, and, in the testis, pregnenolone is a precursor to testosterone.

Memory enhancement has been observed in aged animals when given pregnenolone or pregnenolone sulfate. Pregnenolone sulfate is both a gamma-aminobutyrate (GABA) antagonist and a positive allosteric modulator at the N-methyl-D-aspartate (NMDA) receptor and may reinforce neurotransmitter systems that may decline with age.

Pregnenolone sulfate was found to stimulate acetylcholine release in the adult rat hippocampus. Acetylcholine release may be due to pregnenolone sulfate's negative modulation of the GABA (A) receptor complex and positive modulation of the NMDA receptor. While a modest increase in acetylcholine release facilities memory processes, elevation of acetylcholine beyond an optimal level is ineffective in doing so.

Also in the treatment of obesity, appetite depressants and/or appetite suppressants have been proposed. These comprise sympathomimetic drugs, canthine hydrochloride, phenylpropanolamine hydrochloride, amfepramone hydrochloride, as well as serotonin-norepinephrine reuptake-inhibitor, like simbutramine (Nakagawa et al. 2000) hydrochloride. All of these substances modify appetite, but as they do not specifically target nucleus arcuate neurones and solely modify their function e.g., via NMDA receptors, antiobesity drugs also effect other than arcuate nucleus structures. This might explain the variety of (side) effects of these substances, apart from just modulating satiety.

However, the popular appetite suppressant drug fenfluramine and dexfenfluramine have been withdrawn from the market. The FDA stated that these two drugs are linked to heart valve disease and Primary Pulmonary Hypertension (PPH). PPH is a rare disease which causes the progressive narrowing of the blood vessels of the lungs and mostly results in death.

Also topiramate has recently been proposed in the treatment of obesity. Topiramate demonstrated appetite suppressant properties. Topiramate belongs to a class of medications called anticonvulsants. Usually it is used with other medications to treat certain types of seizures in patients with epilepsy or Lennox-Gastaut syndrome (a disorder that causes seizures and developmental delays). Accordingly, topiramate, marketed as an anti-epileptic drug, is now being evaluated for other indications like obesity, neuropathic pain and management of bipolar mania (The Pharmaceutical Journal Vol. 263, No 7064, page 475, Sep. 25, 1999).

Furthermore, topiramate improves NMDA receptor hypofunction in schizophrenia.

Specifically, topiramate potentiates GABAergic neurotransmission and antagonizes the excitotoxic actions of glutamate at the alpha-amino-3-hydroxy-5-methylisoxazole-4-propionic acid (AMPA)/kainate (KA) classes of glutamate-gated channels. Topiramate was shown to attenuate the severity of negative symptoms in patients with schizophrenia, however, without showing weight increase which is one of the typical side effects of antipsychotic drugs commonly used (such as quetiapine, risperidone, olanzapine, haloperidol).

As stated in Fujioka, Obes Res. (2002) Suppl 2:116 S-123S topiramate is a structurally and pharmacologically novel anticonvulsant agent that was approved in 1996 for treatment of epilepsy. Unlike most antiepileptic agents, topiramate seems to have positive effects on weight. It may produce appetite suppression by blocking kainate/alpha-amino-3-hydroxy-5-methylisoxozole-4-propionicacid glutamate receptor subtypes, but it also has several other actions, including antagonist of voltage-gated sodium channels and modulation of alpha-aminobutyric acid-A activity. Animal pharmacology studies relevant to weight loss have demonstrated that topiramate can increase energy expenditure and reduce food intake, resulting in decreased energy deposition. These effects were associated with a selective loss of body fat and decreased levels of certain metabolic variables (e.g., leptin, insulin). At the 2002 American Diabetes Association Annual Meeting, Bray et al. presented data on the dose-related effects of topiramate in obese patients. This was a 6-month randomized, placebo-controlled study of ascending doses of topiramate. In the intention-to-treat population, the greatest weight loss was seen in the patients taking the highest dose of topiramate (384 mg vs. placebo, 6.3% vs. 2.6%). Significant weight loss was also seen with lower doses of topiramate: 64 mg/d produced 5.0% weight loss, 96 mg produced 4.8%, and 192 mg produced 6.3% weight loss. For the completers, up to 8.5% weight loss was seen at the 384-mg dose of topiramate, and the weight loss was clearly dose-dependent. Topiramate has been evaluated in other obesity-related diseases, including binge-eating disorder; however, these studies were small and not randomized. Topiramate at doses ranging from 100 to 1400 mg was studied in 13 female patients with binge-eating disorder in an open-label study. The effect of topiramate was dose-related, with seven patients achieving weight loss in excess of 5 kg. Several case reports have also been published showing weight loss with topiramate in patients with binge-eating disorder; in patients gaining excessive weight on antipsychotic agents; and after mood stabilization in obese patients with major depression, bipolar disorder, or psychotic disorder. Although topiramate seems to have a positive effect on weight in the obese patient, it is currently only approved for the treatment of seizure disorders.

However, topiramate is known to provide for side effects in brain regions. The mechanism of action of topiramate is not fully understood, but Kaminski et al. (2004) showed that topiramate selectively inhibits postsynaptic responses mediated by GluR5 kainate receptors, and provided evidence for a unique mechanism of action of topiramate, which involves GluR5 kainate receptors. The anatomical distribution of the various subtypes of glutamate binding sites differs (Greenamyre, 1985). Thus as it is not the GluR5 kainate receptors, but the N-methyl-D-aspartate (NMDA) receptor subtypes that play a major role in mediating excitotoxicity, Topiramate may not be regarded a primary tool for protecting arcuate nucleus neurones from glutamate induced cell damages.

Even if several candidate genes have been associated with human obesity or its metabolic complications, the identification of additional or concise factors that influence obesity and/or adiposity is necessary. Strategies to treat and/or prevent pathological body-weight/body mass regulations are desired.

Therefore, the technical problem underlying this invention was to provide for means and methods for modulating (pathological) metabolic conditions, influencing body-weight regulation, and/or energy homeostatic circuits. The solution to said technical problem is achieved by providing the embodiments characterized in the claims.

Accordingly, the present invention provides for the use of an NMDA receptor antagonist, in particular of memantine or neramexane, or a pharmaceutically acceptable salt or a prodrug of said NMDA receptor antagonists in the preparation of a pharmaceutical composition for the prevention, amelioration and/or treatment of disorders of metabolism influencing body weight, in particular obesity, an eating disorder and/or in the regulation of appetite. Also provided is a method for the prevention, amelioration and/or treatment of disorders of metabolism influencing body weight, in particular obesity, an eating disorder and/or in the regulation of appetite comprising the step of administering a therapeutically effective amount of an NMDA receptor antagonist, in particular of memantine or neramexane, or a pharmaceutically acceptable salt or a prodrug of said NMDA receptor antagonists. The uses and methods provided herein are particularly useful for the prevention, amelioration and/or treatment of disorders of metabolism influencing body weight, in particular obesity, an eating disorder and/or in the regulation of appetite in human subjects/human patients in need of such a prevention, amelioration and/or treatment.

In context of the present invention, the term “NMDA receptor antagonist” or “N-methyl D-aspartate receptor antagonist” relates to compounds which are in vivo and/or in vitro capable to block, either completely or partially, the action and/or function of the NMDA receptor or the NMDA receptor complex. Corresponding examples are well known in the art. For example Danysz and Parsons have described in 2002 in Neurotox Res 4, :119-26 several glutamate receptor antagonists (NMDA receptor antagonists) and their neuroprotective roles. The person skilled in the art is readily in a position to employ such known NMDA (receptor/complex) antagonists. Various NMDA receptor antagonists have been developed for acute and chronic neurodegeneration. Some NMDA (receptor/complex) antagonists block the ion channel, others act at the glycine(B) site. Other substances are selective for NR2B NMDA receptor subtypes (see, inter alia, Danysz and Parsons (2002) Neurotox Res 4, 119-126 or Danysz et al (2002) Curr Pharm Des 8, 835-843). Wood (2005) has summarized the pharmacology of the NMDA-receptor complex in a recent article and has also described useful NMDA receptor antagonists (IDrugs. 8, 229-35). Accordingly, NMDA receptor antagonists are well known and compounds speculated and/or suspected to be NMDA receptor antagonists may be deduced by methods known in the art, like transfection studies (for example in Xenopus oocytes) and/or (electro-) physiological measurements. Also in animal experiments the usefulness and the toxicity of a potential NMDA receptor antagonist may be measured by methods known in the art (see also common methods described, inter alia, in Kandel, Schwartz, Jessel, “Principles of Neuronal Science”, 4^{th} edition, 2000). Recently, Kiss (2005) Neurochem Int. 46, 453-464 has also characterized a novel NR2B selective NMDA receptor antagonist employing commonly known techniques like (whole-cell configuration of the) patch clamp technique, calcium flux, and radioligand binding.

Particular preferred NMDA receptor antagonists are defined herein below and also corresponding examples are provided in the experimental part. Accordingly, these antagonists (as defined herein e.g. memantine) can serve as “positive control” in the elucidation and/or verification of a given NMDA receptor antagonist.

The term “NMDA receptor antagonist” as employed herein relates to substances which can negatively modulate NMDA receptors/NMDA complexes. Said “negative modulation” comprises an inhibition and/or blockage. Said inhibition and/or blockage may be partial or complete. NMDA receptors/complexes are well known in the art and, inter alia, described in Kandel, Schwartz, Jessel (2000, loc.cit.). Dingledine and Conn (2000) describe the classes of ionotropic glutamate receptors and also classify the ionotropic glutamate receptor subtype “NMDA receptor”. Accordingly, the person skilled in the art is readily in a position to deduce and test potential NMDA receptor antagonists to be employed in context of the present invention. The antagonist to be employed in context of this invention, most preferably, inhibits/blocks (an) human NMDA receptor(s). Human NMDA receptors have been described in the art and are also described in their protein structure and/or their encoding nucleotide sequences. Corresponding sequences may easily be obtained in current databases, like the EMBL-EBI database under www.ebi.ac.uk or the NCBI database under www.ncbi.nlm.nih.gov. Exemplified, non limiting NMDA receptors comprise the receptors encoded by nucleotide sequences as shown under NM_{—}00835 and NM_{—}00833 in the NCBI database (gene accession number). Further non-limiting gene accessions comprise Genebank A38680, D13515, L05666, L13266-8, or U08107.

As known in the art, uncoordinated, tonic stimulation of NMDA receptors produces enhanced synaptic noise and impairs the ability of the synapse to recognise and transmit physiological signals. This is characteristic of patients suffering e.g., from dementia and Alzheimer's disease, and can successfully been treated with NMDA receptor antagonists. Memantine is a low to moderate affinity, non-competitive NMDA receptor antagonist with strong voltage dependency and rapid blocking/unblocking kinetics that is clinically well-tolerated. Memantine has extensively been tested in clinical studies (see Möbius et al. (2004) Drugs of Today 40, 685-695; and Johnson, Kotermanski (2006) Curr Opinion Pharm 6:61-67). Memantine blocks the sustained activation of NMDA receptors by μM concentrations of GLU under pathological conditions, but rapidly leaves the NMDA receptor channel upon transient physiological activation by low mM concentrations of synaptic GLU (Clements et al., 1992 Science 258(5087):1498-501; Parsons et al. 1993, Neuropharmacology 32:1337-50).

Yet, it is envisaged that in the herein described uses and methods NMDA receptor antagonist are employed which have less side effects than others. Accordingly, in most preferred embodiment of the uses and methods provided herein, substances like memantine, neramexane (MRZ 2/579), licostinel (ACEA 1021), CP-101606, Co101244 and eliprodil (or pharmaceutically acceptable salts or prodrugs of these substances) are employed. Neramexane (MRZ 2/579) has also been proposed to play a role in the pharmacotherapy for alcoholism, see Nagy (2004) Idrugs 7, 339-350. In Nagy (2004. loc. cit.) also further NMDA receptor antagonists are described which may be employed in context of this invention.

Accordingly, and in a more particular preferred embodiment of the invention, 1-amino-alkylcyclohexanes, like memantine or neramexane are envisaged as NMDA receptor antagonist in the uses and methods of this invention are. These 1-amino-alkylcyclohexanes are well known in the art and, inter alia, described in WO 2005/009421 or US 2004/0087658. Neramexane is also known as MRZ 2/579 (1-amino-1,3,3,5,5-pentamethyl-cyclohexan) and CAS-219810-59-0. Accordingly, the invention also relates to the herein described uses and methods whereby MRZ 2/579 and other amino-alkyl-cyclohexanes are employed. As illustrated in the appended examples, in particular memantine is useful in the prevention, amelioration and/or treatment of disorders of the metabolism influencing body weight, in particular in the treatment of obese subjects, in particular in the treatment of human subjects.

Neramexane may, inter alia, be employed in the form of its pharmaceutically acceptable salts, preferably in the form of its hydrochloride salt, i.e. neramexane hydrochloride, namely 1-amino-1,3,3,5,5-pentamethyl-cyclohexan hydrochloride. In another preferred embodiment neramexane may be employed in the form of its mesylate salt, i.e. neramexane mesylate, namely 1-amino-1,3,3,5,5-pentamethyl-cyclohexane mesylate.

Memantine is described in detail herein and further details may be found in U.S. Pat. No. 4,122,193; U.S. Pat. No. 4,273,774 or U.S. Pat. No. 5,061,703. Again, as pointed out herein above all the compounds used in accordance with this invention may also be employed in form of pharmaceutically acceptable salts, like memantine hydrochloride and the like.

As illustrated in the appended examples, memantine is particularly useful in context of this invention and, accordingly, preferred NMDA receptor antagonist to be employed in the methods and uses provided herein are 1-aminocyclohexane derivatives, like memantine and neramexane. In particular in context of 1-aminocyclohexane derivatives, also optical isomers, diasteromers, enantiomers, hydrates, N-methyl, N,N-dimethyl, N-ethyl and N-propyl derivatives and pharmaceutically acceptable salts thereof and mixtures of any of the foregoing are envisaged in context of this invention.

Apart from the glutamate gated ion channel blocker memantine that has been clinically used for many years, other amino-alkyl-cyclohexane derivatives have been evaluated in vitro and in animal models, of which neramexane HCl (MRZ 2/579) was selected for further development (Danysz et al. (2002, loc.cit.). This agent shows some similarity to memantine e.g. channel blocking kinetics, voltage dependency, and affinity. Preclinical tests indicated particularly good activity in animal models of alcoholism and pain. Neramexane exhibits similarly excellent safety and tolerability as memantine, and is, accordingly, also a particular preferred NMDA receptor antagonist to be employed in context of this invention.

Antagonising the glycine site of the NMDA receptor also offers an approach in blocking the NMDA receptor. Many of the glycine site antagonists were shown to lack most of the side effects, such as memory impairment, ataxia, lack of motor coordination and psychotomimetic effects, which accompanied competitive and non-competitive NMDA receptor antagonists (Petty (2004) CNS Drug Rev. 10, 337-348). To date, much has been done to improve the structure-activity relationship of compounds resulting in the synthesis of ACEA 1021. There are much in vitro and in vivo data to support its neuroprotective effects and improved safety profile. Accordingly, ACEA 1021 is another preferred NMDA receptor antagonist to be employed in context of this invention and being clinically useful in the treatment of metabolic disorders, like obesity. In context of this invention, the term “obesity” also comprises “adipositas” and the terms are used synonymously.

Several substances are selective for the NR2B NMDA receptor subtypes. The NMDAR2B subunit is the focus of increasing interest as a therapeutic target in a wide range of CNS pathologies, including acute and chronic pain, stroke and head trauma, drug-induced dyskinesias, and dementias. The apparent superior preclinical and clinical data is likely to reflect subtype selectivity, a unique mode of action and cellular location of the NR2B receptors in the CNS (Chazot 2004). The NMDA NR2B subunit receptor specific antagonist, CP-101606, dose-dependently improved the rate of functional recovery and protected against the ischemic brain damage. It has been concluded that NMDA NR2B receptor subunits represent potential targets to reduce not only the functional deficits, but also neuronal death in cortex and several midbrain regions produced by cerebral ischemia (Kundrotiene et al. 2004). Also eliprodil antagonises the NR2B subunit. Eliprodil has been shown to protect from NMDA receptor-mediated excitotoxicity during ethanol withdrawal (Thomas et al. 2004). Co101244, another novel potent and selective NR1/2B NMDA receptor antagonist, has been found promising in antiepileptic medication (Kohl and Dannhardt 2001).

Selfotel (CGS 19755), a known competitive N-methyl-D-aspartate receptor antagonist, is neuroprotective in experimental models of ischemic cerebral injury; see, inter alia, Yenari (1998) Clin Neuropharmacol. 21:28-34. Solfotel, in a less preferred embodiment is also envisaged in the uses and methods provided herein as a useful NMDA receptor antagonist.

Sun and colleagues (2004) have recently characterized two novel N-methyl-D-aspartate receptor antagonists: EAA-090 (2-[8,9-dioxo-2,6-diazabicyclo [5.2.0]non-1(7)-en2-yl]ethylphosphonic acid) and EAB-318 (R-alpha-amino-5-chloro-1-(phosphonomethyl)-1H-benzimidazole-2-propanoic acid hydrochloride); see J Pharmacol Exp Ther. 310, 563-70. EAA-090 is also known as perzinfotel; see, inter alia, Brandt (2005), J Pharmacol Exp Ther. published March 16^{th}. Again, these NMDA receptor antagonists are examples of compounds to be employed in context of this invention.

Aptiganel is also known as CERESTAT (4, aptiganel CNS 1102) and was evaluated in clinical trials for the treatment of traumatic brain injury and stroke. Whereas aptiganel appeared not to be very successful in theses trials, it is also envisaged as a potential NMDA receptor antagonist to be employed in accordance with this invention. Kroppenstedt 1998 has reported protective effects of aptiganel HCl (Cerestat) following controlled cortical impact injury in the rat; see J Neurotrauma. 15, 191-197.

Also 6,7-dichloro-5-nitro-1,4-dihydro-2,3-quinoxalinedione (licostinel) is well known in the art as NMDA receptor antagonist and is also known as ACEA 1021.

For example Lingenhohl (1998) describes ACEA 1021 as an antagonist at the strychnine-insensitive glycine site of the N-methyl-D-aspartate receptor; see Neuropharmacology 37, 729-737.

Another example of an NMDA receptor antagonist known in the art is gavestinel. In a recent analysis, gavestinel did not improve the outcome after acute intracerebral hemorrhage (see Haley (2005), to be published in Stroke 2005), however may be useful in context of this invention.

Eliprodil is a further NMDA receptor antagonist to be employed in accordance with this invention and is α-(4-Chlorophenyl)-4-[(4-fluorophenyl)methyl]-1-piperidineethanol. Normally, eliprodil is not employed in form of a salt, however, also pharmaceutically acceptable salts and prodrugs thereof are also envisaged in the uses and methods of this invention. Eliprodil is a more preferred compound to be employed in context of this invention. U.S. Pat. No. 5,547,963 describes eliprodil and its enantiomers. Accordingly, in context of this invention, also enantiomers of eliprodil are useful. Accordingly, eliprodil may be employed in a preferred embodiment in form of an enantiomer, in particular as R-eliprodil. Eliprodil has also been described in WO 97/33582, WO 97/02823, U.S. Pat. No. 5,023,266 and U.S. Pat. No. 5,547,963.

In context of this invention, the NMDA receptor antagonists may also be employed in form of optical isomers, diasteromers, enantiomers and the like, as documented herein above. The term “NMDA receptor antagonist” as employed herein, comprises competitive NMDA receptor antagonists (like selfotel) and non-competitive NMDA receptor antagonists (like dextrorphan, GV150526, aptiganel and eliprodil).

Several substances selective for NR2B NMDA receptor subtypes such as CP-101606 and Ro-25-6981 have been shown to have a good neuroprotective profile; see Danysz and Parsons (2002) Neurotox Res. 4, 119-126. Also these compounds, i.e. CP-101606 and Ro-25-6981 (or pharmaceutically acceptable salts thereof or prodrugs thereof) may be employed in context of this invention. CP 101606 (also CP-101,606) was discussed in Kundrotiene (2004) Neurotrauma. 21, 83-93.

Another preferred NMDA receptor antagonist to be employed in context of this invention is, e.g. Co101244, a novel potent and selective NR1/2B NMDA receptor antagonist, described, inter alia, in Kohl (2001) Curr Med. Chem. 8, 1275-1289. Kohl (2001, loc.cit.) also describes further NMDA receptor antagonists in preclinical and biological testing (like dizocilpine, conantokins, Co101244/PD174494, ifenprodil, arcaine, L-701,324, CGP40116, LY235959, LY233053, MRZ2/576, LU73068, 4-Cl-KYN) which may be considered of particular pharmaceutical interest in the context of the present invention, i.e. to be used in the methods and pharmaceutical uses provided herein or which may be employed in academic studies, also animal experiments, relating to disorders of the metabolism and/or body weight disorders (obesity).

As documented herein above, memantine and neramexane are the most preferred NMDA receptor antagonists to be employed in context of this invention. Memantine is the first representative of a new class of Alzheimer drugs—a moderate affinity NMDA-receptor antagonist. Memantine has been developed by Merz Pharmaceuticals and was recently approved in Europe and the USA for the treatment of moderate to severe Alzheimer's disease.

Memantine is well known in the art and, inter alia, described in U.S. Pat. No. 3,391,542; Gerzon (1963), J. Med. Chem. 6, 760 or WO 2004/112758 (describing a particular pharmaceutical dosage form). In US 2005/031652 an aqueous-based carrier comprising also memantine is described. Memantine is also known from Kleemann/Engel (4^{th} edition, 2001; Thieme Verlag). The chemical structure (formula I) of memantine is

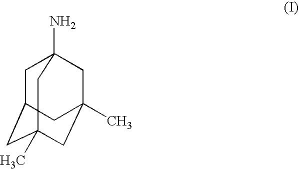

The most common pharmaceutical salt employed in patients, in particular human patients is the hydrochloride form, i.e. memantine hydrochloride. Memantine hydrochloride is also a preferred salt in context of this invention.

Neramexane is a drug that also blocks the effects of excessive glutamate at the NMDA receptor. Neramexane was investigated as monotherapy in patients with moderate to severe dementia of Alzheimer's Type. Neramexane is, moreover, studied for the treatment of serious neurological and psychiatric diseases. Neramexane was also developed by Merz Pharmaceuticals.

Neramexane (1-amino-1,3,3,5,5-pentamethylcyclohexane) is well known in the art and, inter alia, described in U.S. Pat. No. 6,034,134. In US 2006 00 2999 immediate release formulations comprising among others neramexane are described. The chemical structure (formula II) of neramexane is

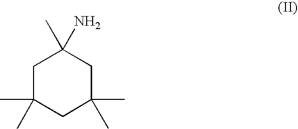

The most common pharmaceutical salt employed in patients, in particular in human patients is the mesylate form, i.e. neramexane mesylate. Neramexane mesylate is also a preferred salt in context of this invention. Accordingly, most preferred NMDA receptor antagonists of the present invention are in the form of their hydrochloride or mesylate salts.

Yet, also other salts are known and envisaged. These comprise, but are not limited to acid addition salts, like acetate, adipate, alginate, ascorbate, aspartate, benzoate, benzenesulfonate, bisulphate, borate, butyrate, citrate, camphorate, camphersulfonate, cyclopentanepropionate, digluconate, dodecyl sulphate, ethane sulfonate, fumarate, glucoheptanoate, glycerophosphate, hemisulphate, heptanoate, hexanoate, hydrochloride, hydrobromide, hydroiodide, 2-hydroxyethane sulfonate, lactate, maleate, methane, mesylate, sulfonate, 2-naphthalene sulfonate, nicotinate, nitrate, oxalate, pamoate, pectinate, persulphate, 3-phenyl sulfonate, 3-phenylpropionate, phosphate, picrate, pivalate, propionate, salicylate, succinate, sulphate, sulfonate, tartrate, thiocyanate, toluene sulfonate such as tosylate, undecanoate, or the like.

In accordance with the invention, also a pharmaceutically active prodrug of the NMDA receptor antagonist to be employed in context of this invention, may be used. As used herein the term “prodrug” refers to (i) an inactive form of a drug that exerts its effects after metabolic processes within the body convert it to a usable or active form, or (ii) a substance that gives rise to a pharmacologically active metabolite, although not itself active (i.e. an inactive precursor). The term “memantine” as employed herein also comprises modifications of the molecules as documented herein. Said “2^{nd} generation memantine” are inter alia demonstrated in Lipton, J. Alzheimers Disease, 6. Suppl. 6, (2004) 61-74.

Memantine, as mentioned above, has been proposed and/or marketed for the treatment and/or prevention of Alzheimer's disease or Parkinson's disease, neramexane for the treatment of serious neurological and psychiatric diseases. Other NMDA receptor antagonists have been proposed in the medical intervention of stroke, alcoholism, and in further neurological disorders, like dementia, ischemic events, closed-head trauma, brain injuries, retinal injury, schizophrenia or epileptic events.

In contrast, the present invention relates to the use of NMDA receptor antagonists, in particular of memantine or neramexane, a pharmaceutically acceptable salt or a prodrug of NMDA receptor antagonists in the medical intervention of a disorder of the metabolism influencing body weight, whereby said disorder of metabolism leads to an (pathologically) increased body weight, like obesity/adipositas. Accordingly, the present invention also proposes to employ NMDA receptor antagonists (or a pharmaceutically acceptable salt) in the regulation of appetite, in particular as a suppressor/depressor of appetite.

The most common disorder of metabolism to be treated, prevented and/or ameliorated in accordance with this invention is obesity/adipositas and/or a disorder which involves higher levels of triglycerides in the blood of a patient to be treated. The recommended level of triglycerides (in a normal range) are in males 40-160 mg/dL and in females 35 to 135 mg/dL. However, in Germany also “higher levels” are tolerated on being normal; e.g. 250 mg/dL. Accordingly, higher levels of triglycerides to be treated with NMDA receptor antagonists, in particular of memantine or neramexane, a pharmaceutically acceptable salt or a prodrug of NMDA receptor antagonists are preferably above 150 mg/dL, more preferably above 200 mg/dL and most preferably above 250 mg/dL.

As documented in the examples, obesity and/or pathological body weight increase result in the first place from voracity, inter alia caused by supraphysiological glutamate consumption. Therefore, the present invention provides for means and methods for the medical intervention in overweight subject, in particular human patients.

An “overweight” patient is often defined as having a body mass index (BMI) above 25 kg/m². Accordingly, the patients to be treated in accordance with this invention have a body mass index between 25 to 30 kg/m². However, it is also envisaged that patients are to be treated who have a BMI above 30 kg/m². In certain medically indicated cases, it is also envisaged that patients with a BMI below 25 kg/m² are to be treated with NMDA receptor antagonists, in particular of memantine or neramexane, a pharmaceutically acceptable salt or a prodrug of NMDA receptor antagonists in order to reduce their body weight.

Accordingly, the present invention provides for new medical use of NMDA receptor antagonists, in particular of memantine or neramexane, a pharmaceutically acceptable salt or a prodrug of NMDA receptor antagonists for preventing or treating obesity, adipositas, eating disorders leading to increased body weight/body mass. Also envisaged are disorders related to higher or pathologically high body weight due to the use of drugs (like corticosteroids, antipsychotic drugs, antidepressants, particularly tricyclic antidepressants, oral contraceptives, etc.).

Disorders of the metabolism linked to higher body weight/body mass and to be treated (or prevented) by the administration of memantine may also comprise, but are not limited to, glycogen storage diseases, lipid storage diseases (like, e.g., Gaucher, Niemann Pick), endocrine disorders (like, e.g., Cushings, hypothyroidism, insulinomas, lack of growth hormone, diabetes, adrenogenital syndrome, diseases of the adrenal cortex), tumors and metastases (such as craniophryngeomas), Prader-Willi syndrome, Down syndrome and genetic diseases and syndromes (like, e.g., hyperlipoproteinemias, hypothalmic disorders, Fröhlich syndrome or empty sella syndrome).

Therefore, the invention also relates to the use NMDA receptor antagonists, in particular of memantine or neramexane, a pharmaceutically acceptable salt or a prodrug of NMDA receptor antagonists in the treatment or prevention of diseases/disorders related to, caused by or leading to higher or pathologically high body weight.

In accordance with this invention it is also envisaged that NMDA receptor antagonists, in particular of memantine or neramexane, a pharmaceutically acceptable salt or a prodrug of NMDA receptor antagonists is employed in the medical intervention of secondary disorders related to a (pathological) increase of body weight. These “secondary disorders” may comprise, but are not limited to diabetes type 2, high blood pressure (hypertension), cardiovascular diseases, cancer, problems with sexual function and disorder of the muscular or bone system.

Problems with sexual function may comprise libido problems, penile dysfunction as well as FSAD (Female Sexual Arousal Disorder). Also dyslipidaemia may be a “secondary disorder” for the treatment by the use of NMDA receptor antagonists, in particular of memantine or neramexane, a pharmaceutically acceptable salt or a prodrug of NMDA receptor antagonists. Also envisaged are growth hormone deficiency, partial growth hormone deficiency or neuro-secretory dysfunction of growth hormone secretion.

The pharmaceutical compositions described herein can be administered to the subject at a suitable dose. Administration of the suitable compositions may be effected by different ways, e.g., by intravenous, intraperitoneal, subcutaneous, as well as transdermal administration.

The NMDA receptor antagonists, in particular of memantine or neramexane, a pharmaceutically acceptable salt or a prodrug of NMDA receptor antagonists may, accordingly, be administered orally, parenterally, such as subcutaneously, intravenously, intramuscularly, intraperitoneally, intrathecally, transdermally, transmucosally, subdurally, locally or topically via iontopheresis, sublingually, by inhalation spray, aerosol or rectally and the like in dosage unit formulations optionally comprising conventional pharmaceutically acceptable excipients.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Furthermore, the pharmaceutical composition described herein may comprise further agents depending on the intended use of the pharmaceutical composition.

It will be appreciated by the person of ordinary skill in the art that the compounds of the invention and the additional therapeutic agent may be formulated in one single dosage form, or may be present in separate dosage forms and may be either administered concomitantly (i.e. at the same time) or sequentially.

The pharmaceutical compositions comprising NMDA receptor antagonists, in particular of memantine or neramexane, a pharmaceutically acceptable salt or a prodrug of NMDA receptor antagonists may be in any form suitable for the intended method of administration. Recently, a novel form of an oral administrable NMDA receptor antagonist (memantine) has been described; see US 2004/25451.

Pharmaceutically useful excipients that may be used in the formulation of the pharmaceutical compositions comprising NMDA receptor antagonists, in particular of memantine or neramexane, a pharmaceutically acceptable salt or a prodrug of NMDA receptor antagonists may comprise carriers, vehicles, diluents, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate; binders, adjuvants, solubilizers, thickening agents, stabilizers, disintegrants, glidants, lubricating agents, buffering agents, emulsifiers, wetting agents, suspending agents, sweetening agents, colourants, flavours, coating agents, preservatives, antioxidants, processing agents, drug delivery modifiers and enhancers such as calcium phosphate, magnesium state, talc, monosaccharides, disaccharides, starch, gelatine, cellulose, methylcellulose, sodium carboxymethyl cellulose, dextrose, hydroxypropyl-B-cyclodextrin, polyvinylpyrrolidone, low melting waxes, ion exchange resins.

Other suitable pharmaceutically acceptable excipients are described in Remington's Pharmaceutical Sciences, 15^{th} Ed., Mack Publishing Co., New Jersey (1991).

Dosage forms for oral administration include tablets, capsules, lozenges, pills, wafers, granules, oral liquids such as syrups, suspensions, solutions, emulsions, powder for reconstitution.

Dosage forms for parenteral administration include aqueous or olageous solutions or emulsions for infusion, aqueous or olageous solutions, suspensions or emulsions for injection pre-filled syringes, and/or powders for reconstitution.

Dosage forms for local/topical administration comprise insufflations, aerosols, metered aerosols, transdermal therapeutic systems, medicated patches, rectal suppositories, and/or ovula.

The amount of NMDA receptor antagonists, in particular of memantine or neramexane, a pharmaceutically acceptable salt or a prodrug of NMDA receptor antagonists that may be combined with the excipients to formulate a single dosage form will vary upon the host treated and the particular mode of administration.

The pharmaceutical compositions of the invention can be produced in a manner known per se to the skilled person as described, for example, in Remington's Pharmaceutical Sciences, 15^{th} Ed., Mack Publishing Co., New Jersey (1991).

For the purpose of the present invention, a therapeutically effective dosage of NMDA receptor antagonists, in particular of memantine or neramexane, a pharmaceutically acceptable salt or a prodrug of NMDA receptor antagonists will generally be from about 2.5 to 100 mg/day, preferably from about 5 to about 50 mg/day, and most preferably from about 10 to about 30 mg/day, which may be administered in one or multiple doses. In a most preferred administration scheme, the administration of about 5 to 50 mg/day, preferably from about 10 to 30 mg/day is envisaged, which may be administered in one or two doses, preferably ⅓ of the daily doses in the morning, and ⅔ of the daily doses late in the afternoon. Corresponding schemes are also illustrated in the appended examples and in the appended figures.

First short clinical experiences in otherwise healthy, but obese patients suggested a dose regimen with memantine 5 to 10 mg in the morning, and 5 to 30 mg late in the afternoon. The patients experienced an initial loss of body weight of 1 to 3 kg within a 7 day period. Further weight loss is observed during the course of administration over a longer period of time, see appended FIG. 6. They reported on significant decrease in appetite, in particular, they reported that the usual late afternoon or evening binge-eating disorders had complete disappeared, already at the second day of treatment. Apart from short moments of mild dizziness at the first day, and mild constipation, there were no side effects during the initial treatment period.

It will be appreciated, however, that specific dose level of the compounds of the invention for any particular patient will depend on a variety of factors such as age, sex, body weight, general health condition, diet, individual response of the patient to be treated time of administration, severity of the disease to be treated, the activity of particular compound applied, dosage form, mode of application and concomitant medication. The therapeutically effective amount for a given situation will readily be determined by routine experimentation and is within the skills and judgement of the ordinary clinician or physician.

The present invention documents experimentally that obesity, voracity and growth hormone deficiency are linked to the consumption of elevated amounts of the amino acid glutamate (GLU). Supraphysiological doses of GLU can enter the lateral hypothalamus, and may interfere with the physiology of appetite regulation, and they may be toxic for neuronal cells of the arcuate nucleus.

It is documented in the appended examples that human data were obtained and analyzed from 807,592 German conscripts born between 1974 and 1978, and from 1,432,368 women of the German birth statistics (deutsche Perinatalerhebung) 1995-1997.

In the experimental part of this invention, the effects of orally administered monosodium glutamate (MSG), were investigated in 30 pregnant Wistar rats and their offspring. Pregnant animals either received no extra MSG, or 2.5 g MSG or 5 g MSG per day, up to the end of the weaning period. 2.5 g, respectively 5 g MSG accounted for some 10%, respectively 20% of dry weight of the average daily food ration. After weaning, MSG feeding was continued in the offspring.

It is of particular interest that in accordance with this invention morbid obesity associates with short stature. Furthermore, average stature of conscripts progressively declines when BMI increases above 38 kg/m². Also morbidly obese young women are shorter than average.

As shown herein, oral administration of MSG to pregnant rats affects birth weight of the offspring. Maternal feeding with 5 g MSG per day results in severe birth weight reduction (p<0.01). Weight increments remain subnormal when MSG feeding to the mothers is maintained during weaning p<0.01). GH serum levels are affected in animals that received MSG during prenatal life via maternal feeding. Animals that are kept on high MSG diet (5 g MSG per day) continue to show serum GH levels that are as low or even lower than those of MSG injected animals (p<0.05), both at day 30 and at day 90 of life. Animals that were kept on medium MSG diet (2.5 g MSG per day) showed low serum GH levels at day 30 of life (p<0.01), but seemed to partially recover before day 90. Almost identical results were observed in IGF-1 serum levels. Oral MSG resulted in dose dependent voracity. The animals fed 5 g MSG per day increased water uptake by threefold (p<0.01), and food uptake by almost twofold (p<0.01).

Most importantly, GLU is a widely used nutritional substance that potentially exhibits significant neuronal toxicity. Voracity, and impaired growth hormone (GH) secretion are the two major characteristics of parenterally administered GLU-induced neuronal damage. GLU maintains its toxicity in animals even when administered orally.

The present invention demonstrates that a widely used nutritional mono-substance—the flavouring agent monosodium glutamate—at concentrations that only slightly surpass those found in everyday human food exhibits significant potential for damaging the hypothalamic regulation of appetite, and thereby determines the propensity of world-wide obesity. In accordance with this invention, daily allowances of amino acids and nutritional protein should be reconsidered and it is strongly recommended to abstain from the popular protein-rich diets and particularly from adding the flavouring agent monosodium glutamate.

However, this invention also provides the basis for the important finding that NMDA receptor antagonists, in particular of memantine or neramexane, a pharmaceutically acceptable salt or a prodrug of NMDA receptor antagonists may be employed in the prevention, treatment and/or amelioration of metabolic disease, in particular metabolic diseases related to high triglyceride levels in the blood of the corresponding subjects patients). Most preferably memantine or neramexane are employed in accordance with this invention in the treatment or prevention of obesity or in the treatment of food-intake disorders or as an appetite suppressor/depressor.

The study presented herein above and demonstrated in the examples below was undertaken to further investigate the links between obesity, voracity, and growth hormone deficiency. Novel human data are presented supporting evidence that morbid obesity not only associates with GH secretory dysfunction but also with short stature. Most importantly, further data provide for supporting evidence that GLU toxicity is not limited to parenteral administration of this amino acid but that oral administration of GLU also causes voracity and GH deficiency, leading to obese phenotypes.

### ADDITIONAL LITERATURE

Banks W A (2003), Curr Pharm Design. 9:801-809.
Berthoud H R (2004), Phys Behav 81:781-793.
Bray G A (1985), Brain Res Bull 14:505-510.
Currie P J, Chang N, Luo S, Anderson G H (1995), Life Sci 57:1911-23.
Duva M A, Tomkins E M, Moranda L M, Kaplan R, Sukhaseum A, Jimenez A, Stanley B G (2001), Brain Res 921:122-132.
Duva M A, Tomkins E M, Moranda L M, Kaplan R, Sukhaseum A, Stanley B G (2005), Neurosci Res 52:95-106.
Hermanussen M, García A P, Sunder M, Voigt M, Salazar V, Tresguerres J A F (2005), Eur J Clin Nutr (advance online publication 30.8.2005).
Johnson J W, Kotermanski S E (2006), Curr Opin Pharmacol 6:61-7.
Khan A M, Curras M C, Dao J, Jamal F A, Turkowski C A, Goel R K, Gillard E R, Wolfsohn S D, Stanley B G (1999), Am J Physiol 276:R880-91.
Monno A, Vezzani A, Bastone A, Salmona M, Garattini S (1995), Neurosci Lett 193:45-8.
Nakagawa T, Ukai K, Ohyama T, Gomita Y, Okamura H (2000), Exp Anim 49:239-49.
Stanley B G, Willett V L, Donias H W, Dee M G, Duva M A (1996), Am J Physiol 270:R443-9.

### THE FIGURES SHOW

FIG. 1. Average body height of 807,592 German conscripts born between 1974 and 1978, aged 19 years, and 1,432,368 young German women at the beginning of pregnancy (deutsche Perinatalerhebung) 1995-1997, versus BMI.

FIG. 2a. Mean (±SEM) birth weight in normal rats (control), neonatally MSG-treated rats (injection) and 2.5 g (2.5 g MSG) and 5 g (5 g MSG) MSG oral administered rats (n=12-18). Each group includes male and female data since statistical analysis showed no gender differences. ** p<0.01 vs other groups.

FIG. 2b. Mean (±SEM) weaning body weight in normal rats (control), neonatally MSG-treated rats (injection) and 2.5 g (2.5 g MSG) and 5 g (5 g MSG) MSG oral administered rats (n=12-18). Each group includes male and female data since statistical analysis showed no gender differences. ** p<0.01 vs CONTROL+INJECTION groups and # p<0.05 vs 2.5 g MSG group.

FIG. 3a. Mean (±SEM) plasma concentration of GH at 30 days of life in normal rats (control), neonatally MSG-treated rats (injection) and 2.5 g (2.5 g MSG) and 5 g (5 g MSG) MSG oral administered rats (n=12-18). Each group includes male and female data since statistical analysis showed no gender differences. ** p<0.01 vs CONTROL group and # p<0.05 vs CONTROL group.

FIG. 3b. Mean (±SEM) plasma concentration of GH at 90 days of life in normal rats (control), neonatally MSG-treated rats (injection) and 2.5 g (2.5 g MSG) and 5 g (5 g MSG) MSG oral administered rats (n=6-9).

* p<0.05 vs CONTROL+2.5 g MSG and ## p<0.01 vs the corresponding FEMALE group.

FIG. 4a. Mean (±SEM) IGF-1 plasma concentration at 30 days of life in normal rats (control), neonatally MSG-treated rats (injection) and 2.5 g (2.5 g MSG) and 5 g (5 g MSG) MSG oral administered rats (n=12-18). Each group includes male and female data since statistical analysis showed no gender differences. ** p<0.01 vs CONTROL group.

FIG. 4b. Mean (±SEM) IGF-1 plasma concentration at 90 days of life in normal rats (control), neonatally MSG-treated rats (injection) and 2.5 g (2.5 g MSG) and 5 g (5 g MSG) MSG oral administered rats (n=6-9).

** p<0.01 vs CONTROL+2.5 g MSG and ## p<0.01 vs the corresponding FEMALE group.

FIG. 5a. Mean (±SEM) water uptake at 90 days of life in normal rats (control), neonatally MSG-treated rats (injection) and 2.5 g (2.5 g MSG) and 5 g (5 g MSG) MSG oral administered rats (n=6-9). ** p<0.05 vs CONTROL+INJECTION groups; † p<0.05 vs INJECTION group; ‡‡ p<0.01 vs 2.5 g MSG group and ## p <0.01 vs the corresponding FEMALE group.

FIG. 5b. Mean (±SEM) food uptake at 90 days of life in normal rats (control), neonatally MSG-treated rats (injection) and 2.5 g (2.5 g MSG) and 5 g (5 g MSG) MSG oral administered rats (n=12-18). Each group includes male and female data since statistical analysis showed no gender differences. ** p<0.01 vs CONTROL+INJECTION group.

FIG. 6. Percent body weight decline in seven obese and one overweight subjects (6 females, 2 males) during a two month therapeutical trial with memantine (Axura®, Merz Pharmaceuticals GmbH), 5-10 mg in the morning, and 5-20 mg late in the afternoon (Patient Gr initial body weight 126.6 kg, Sc initial body weight 90.6 kg, Re initial body weight 93.5 kg, Br initial body weight 122.3 kg, Ku initial body weight 111.0 kg, Ho initial body weight 77.6 kg, Sk (male) initial body weight 148.6 kg, Le (male) initial body weight 109.6 kg).

During the first 10 days of treatment, Ku went on eating large portions because she did not want “to waist nutrition”. Br was constipated between day 10 and day 16 of treatment.

### THE EXAMPLES ILLUSTRATE THE INVENTION

### Example 1

### Material and Methods Used and Employed in this Study

### Human Data

Body height and body mass index (BMI) were obtained from 807,592 German conscripts born between 1974 and 1978, aged 19-20 years. The data were given to us by courtesy of the Institut für Wehrmedizinalstatistik und Berichtswesen, Remagen, Germany. All conscripts had either completed high school (A-level, German: Gymnasium), secondary school (O-level, German: Realschule), or 9 year elementary school (German: Hauptschule). We excluded persons who were chronically ill, or lived under the care of a guardian, and conscripts who did not complete school education. This was done on purpose in order to exclude mentally handicapped subjects suffering from Down-Syndrome, Prader-Willi-Syndrome, and other syndromes with short stature and obesity. We assumed that very obese young men had also been obese during the final period of adolescent growth.

Maternal data on body height and weight at the beginning of pregnancy from 1,432,368 women, were obtained from the German birth statistics (deutsche Perinatalerhebung) 1995-1997 (Voigt, Analyse des Neugeborenenkollektivs der Jahre 1995-1997 der Bundesrepublik Deutschland, Geburtsh. Frauenheilk. (2001), 61; 700-706). We rejected adolescent mothers, in order to exclude those who had not yet reached final height, and restricted the sample to persons below the age of 30 years. We assumed that very obese young females had also been obese during the final period of adolescent growth. We are aware that data obtained from birth statistics are not representative for women in general. Pregnancy provides evidence for unimpaired hypothalamic-pituitary-gonadal function. But, large samples of unselected young women are not available in Germany.

### Animal Data

The effects of oral administration of GLU were investigated in 32 pregnant rats and their offspring up to day 90 of life, in the animal facilities of the Department of Physiology, Medical School, Universidad Complutense, Madrid, Spain. The study was conducted in accordance with the principles and procedures outlined in the NIH Guide for the Care and Use of Laboratory Animals. Pregnant Wistar rats were kept under controlled conditions of light (12 h light/12 h darkness) and temperature (21+/−2C.°), and were fed with tap water and rat chow (Panlab, Barcelona, Spain) ad libitum.

At day 14 of pregnancy, the animals were divided into four groups (n=8), and either received no extra monosodium glutamate (MSG) (control, group 1), or 2.5 g MSG (group 2) or 5 g MSG per day (group 3), up to the end of the weaning period. 2.5 g, respectively 5 g MSG accounted for some 10%, respectively 20% of dry weight of the average daily food ration. After weaning and gender separation, MSG feeding was continued in the offspring at the same concentrations. A fourth group of animals received no MSG, but their offspring was injected with MSG 4 mg/g body weight s.c., at neonatal age as described earlier (Hermanussen, (1996) loc. cit.). Due to the existence of two gender groups a total of 8 experimental groups were obtained with n=6-9 for each group of females and males. All litter was weighed at weekly intervals, and the amount of food consumed was registered daily. Offspring was sacrificed half at day 30 and the rest at day 90 of life.

### Tissue Preparation

Half of the animals were killed by decapitation at day 30 and the other half at the end of the observation period at day 90. Anterior pituitaries were removed, and trunk blood was collected. Serum samples and anterior pituitaries after being weighted, were kept at −80° C., for hormonal determinations by specific radioimmunoassays (RIA). Pituitary homogenates were obtained by manual glass homogenizers, and processed in saline after thawing in the moment of the measurement.

### Radioimmunoassays

Plasma GH levels and pituitary GH content were determined by RIA as previously described.²¹ Pituitary homogenates were diluted 1:5000 for determination. Reagents were kindly provided by the National Institute of Diabetes, Digestive and Kidney Diseases (NIDDK). The standard used was rat GH RP2. The sensitivity of the curve was 2 ng/ml and the intraassay coefficient of variation was 5.7%. Plasma IGF I concentrations were measured using a commercially available rat RIA kit (DSL-2900, Diagnostic Systems Laboratories, INC). The sensitivity of the assay was 20 ng/ml, and the intraassay coefficients of variation for mean serum concentrations of 323, 772 and 1604 ng/ml were 5.9%, 6.1% and 3.8%, respectively.

Leptin levels were determined by RIA using a commercial kit (RL-83K, LINCO RESEARCH), with a sensitivity of 0.5 ng/ml, and intraassay coefficients of variation of 2.4% (1.6 ng/ml), 4.1% (3.3 ng/ml), 2% (6.8 ng/ml) and 4.6% (11.6 ng/ml).

### Statistical Analysis

Values are expressed as mean +/−SEM. In some cases data were subject to log transformation since variances showed a log-normal distribution. To determine differences in final weight or hormonal levels a two-way ANOVA test was performed. Differences among groups were subjected to a post-hoc comparison by using Tukey HSD for unequal N test. Statistics were executed using Statistica program. The significance level was determined to be p<0.05.

### Example 2

### Human Data Document that Morbid Obesity Associates with Short Stature

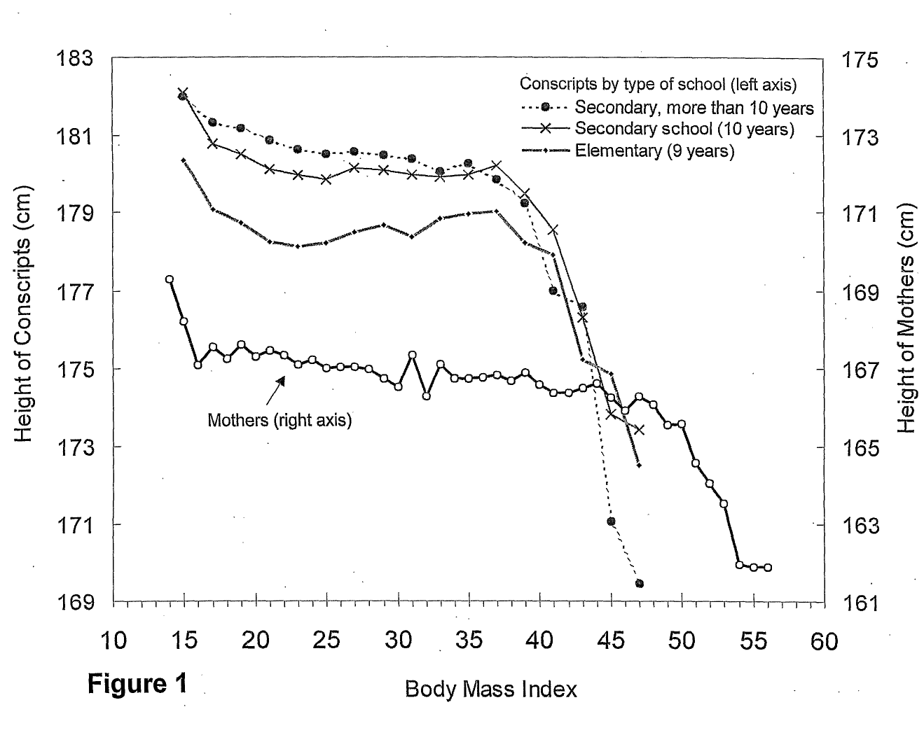

Regardless of school education, average stature of conscripts progressively declines when BMI increases above 38 kg/m². The same applies for fertile women. Morbidly obese young women are shorter than average (FIG. 1).

### Example 3

### Animal Data Document that the Flavouring Agent Monosodium Glutamate Damages Physiological Regulation of Appetite

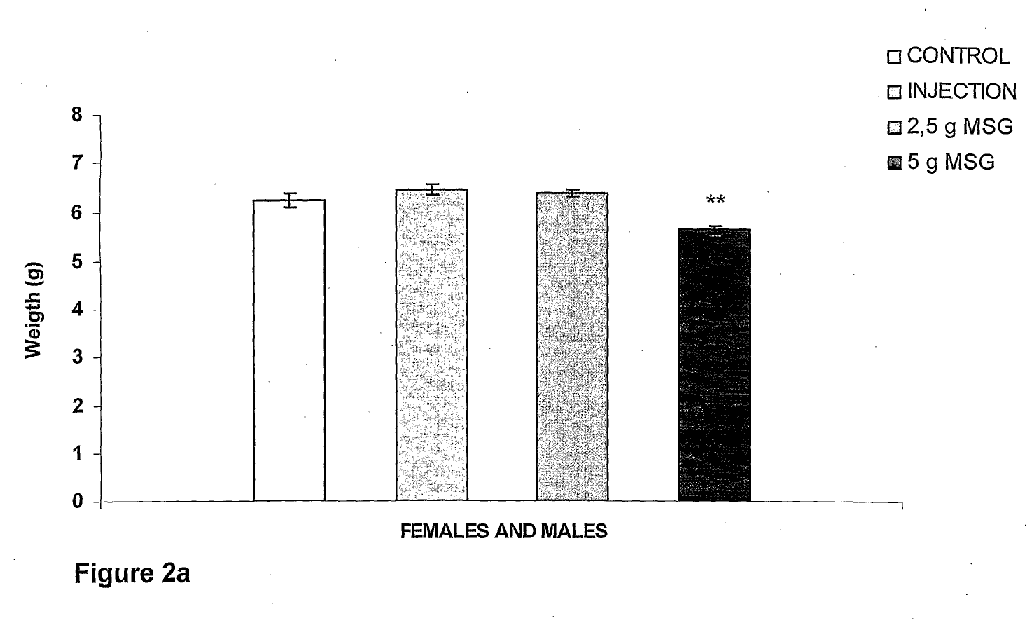

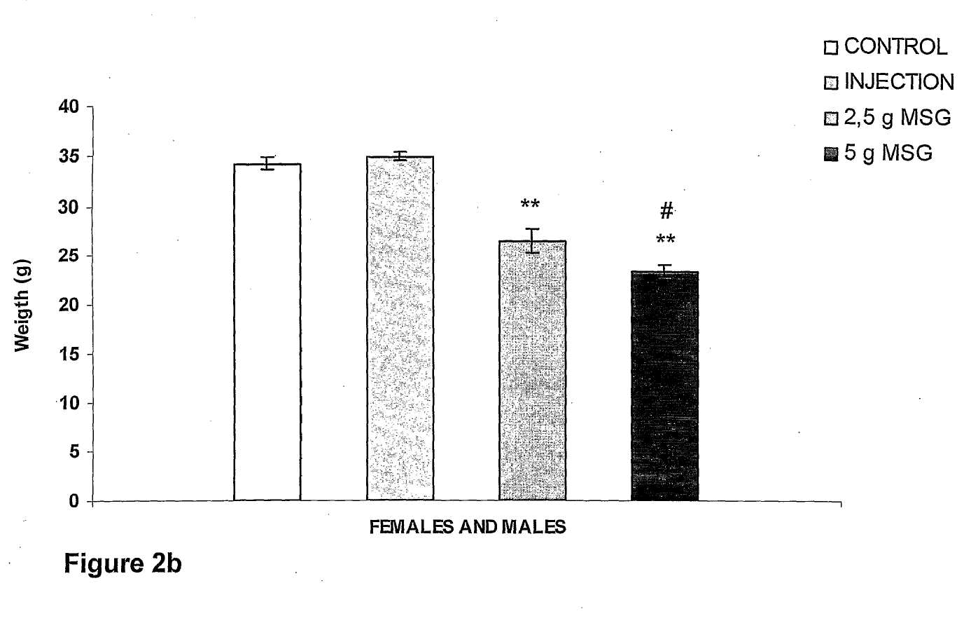

Oral administration of monosodium glutamate (MSG) to pregnant Wistar rats affects birth weight of the offspring (FIG. 2a). Maternal feeding with 2.5 g MSG per day (group 2) results in no birth weight modification as compared to controls, whereas maternal feeding with 5 g MSG per day (group 3) results in severe birth weight reduction (p<0.01). Weight increments remain subnormal when MSG feeding to the mothers is maintained during weaning (FIG. 2b) (p<0.01).

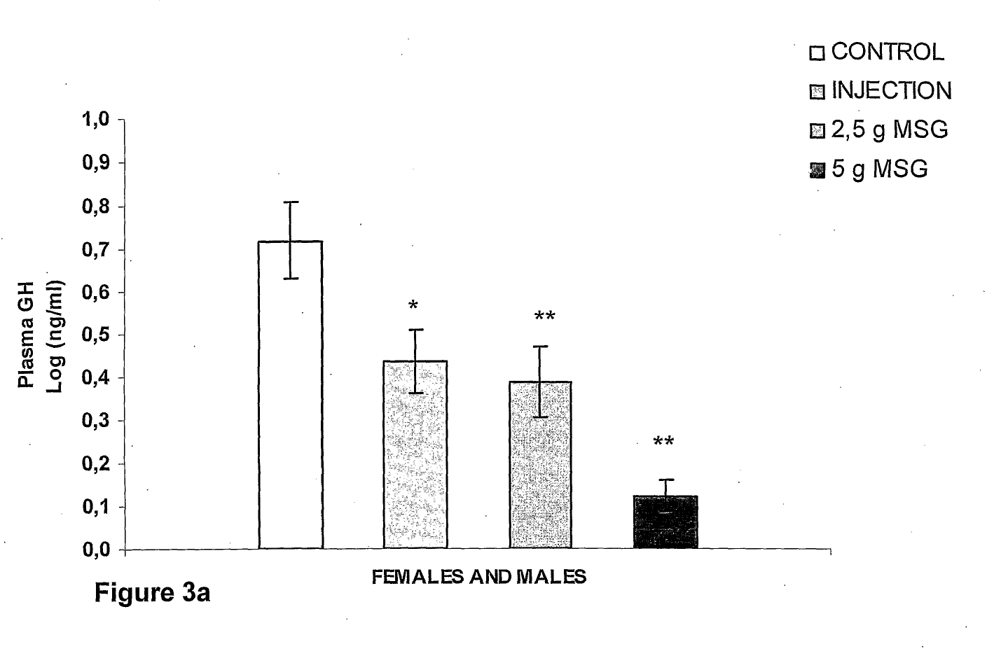

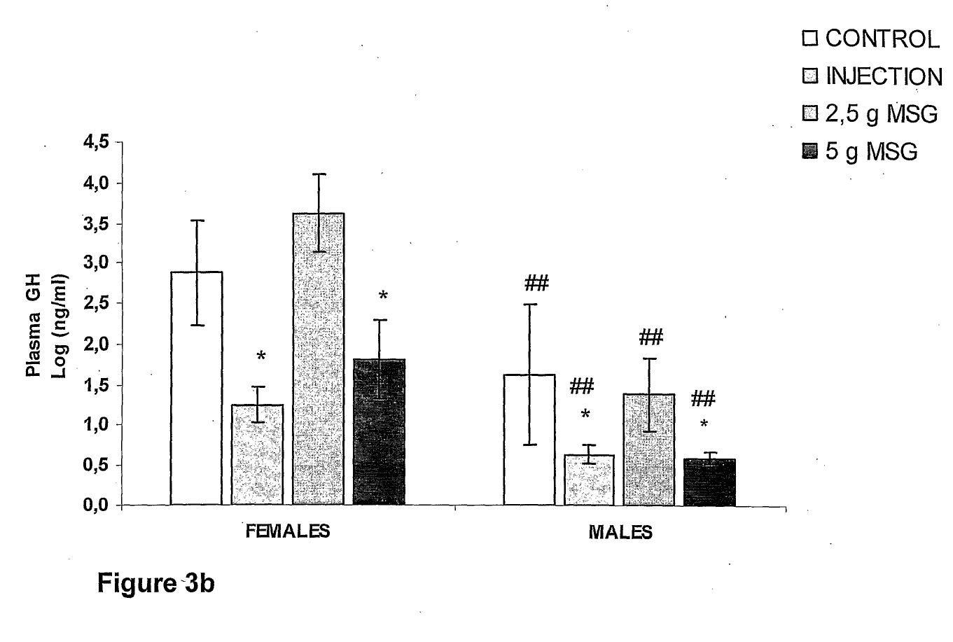

FIG. 3 a/b shows growth hormone (GH) plasma levels of the offspring. As expected, GH plasma levels were low in animals that were neonatally injected with MSG, both at day 30 and at day 90 of life (p<0.05). But GH serum levels were also affected in animals that had received MSG during prenatal life via maternal feeding. FIG. 3b illustrates that animals kept on high MSG diet (5 g MSG per day) show serum GH levels that are as low or even lower than those of MSG injected animals (p<0.05), both at day 30 and at day 90 of life.

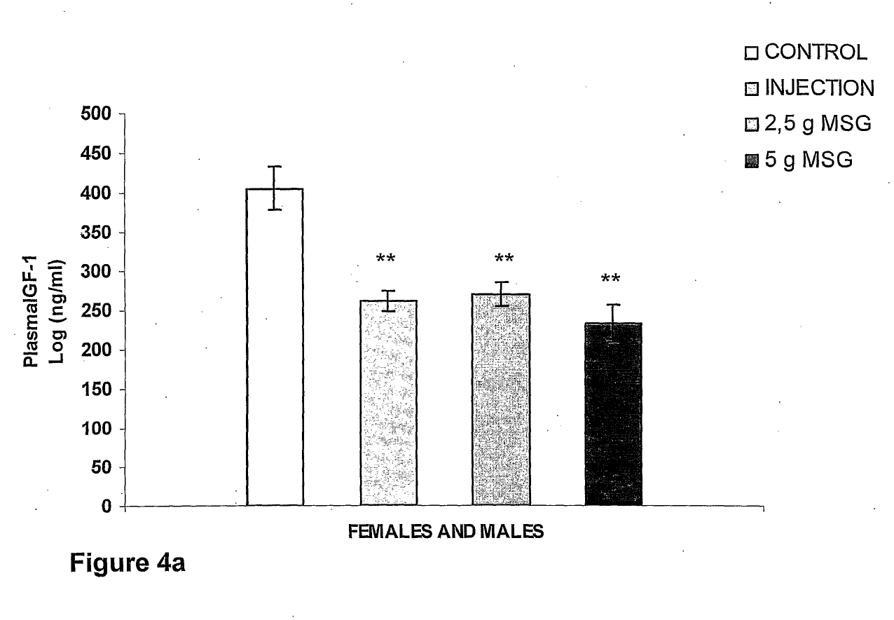

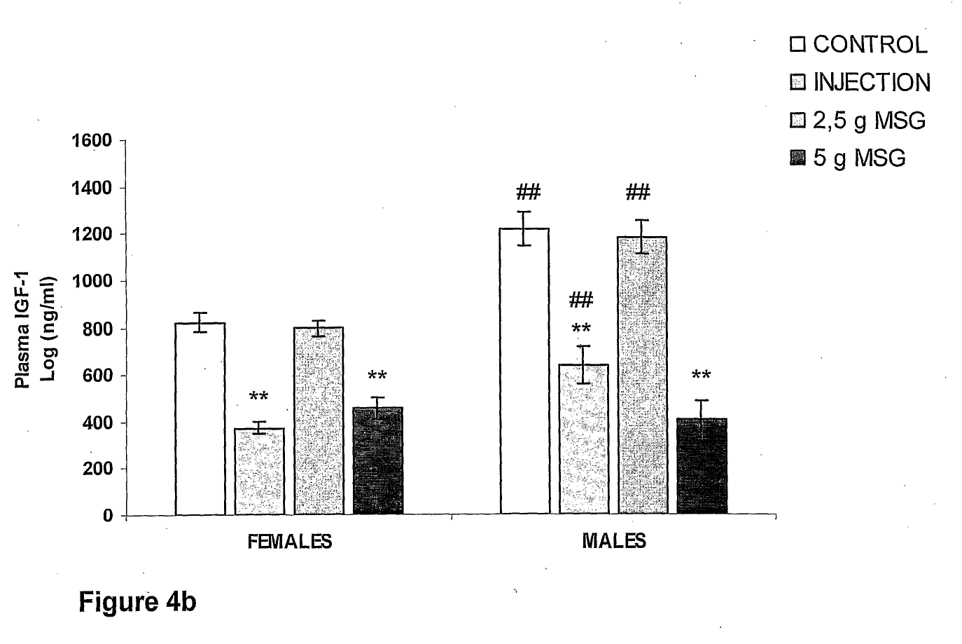

Animals that were kept on medium MSG diet (2.5 g MSG per day) showed low serum GH levels at day 30 of life (p<0.01), but seemed to partially recover before day 90. Almost identical results were observed in IGF-1 serum levels (FIG. 4 a/b).

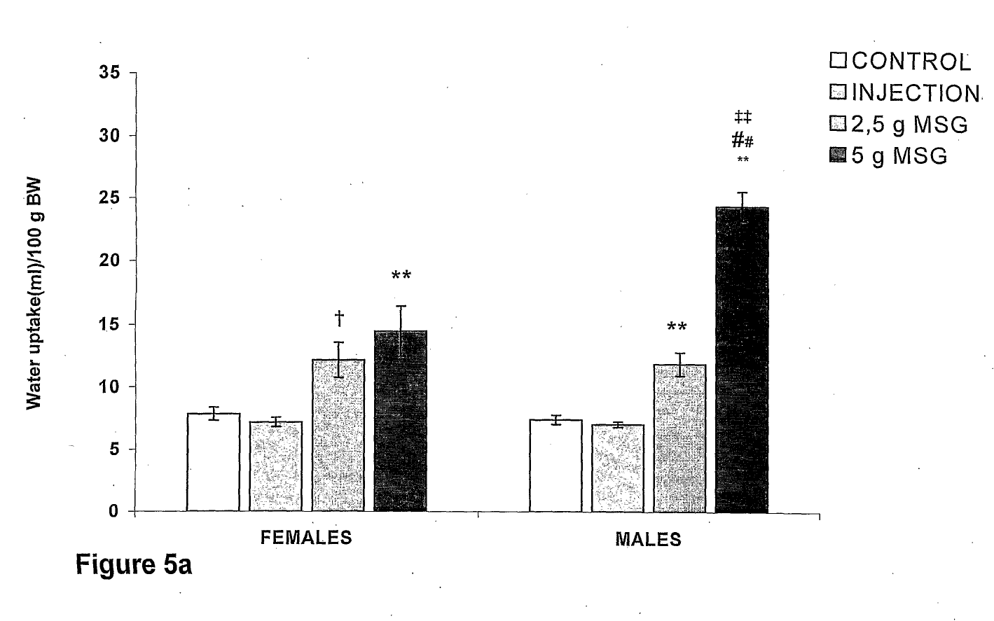

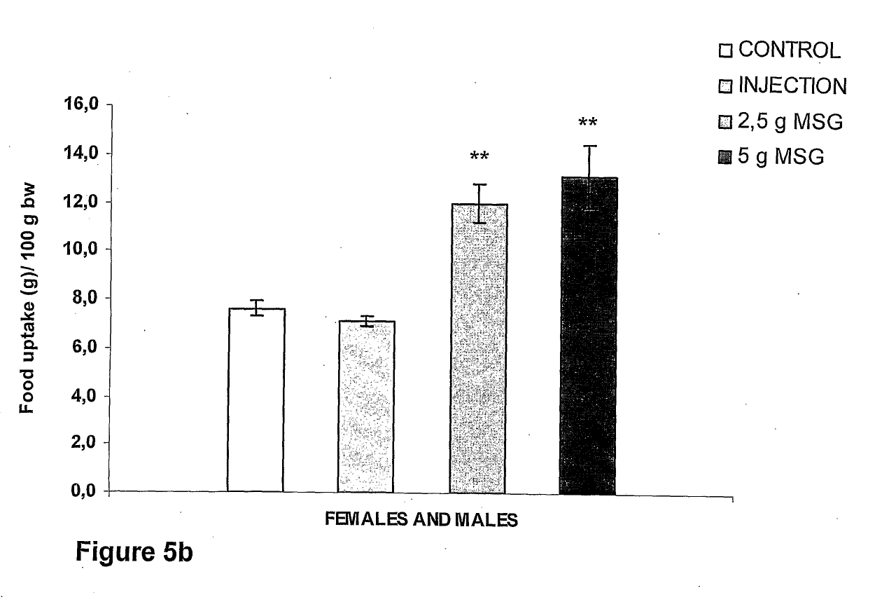

FIG. 5 a/b shows the influence of MSG on appetite. Whereas—in contrast to previous findings (Fan, (1997), loc. cit.)—MSG injected animals of this investigation did not show significantly increased appetite compared to controls, the animals kept on medium MSG diet (2.5 g MSG per day), and particularly those kept on high MSG diet (5 g MSG per day) demonstrated marked voracity. The animals fed 5 g MSG per day increased water uptake by threefold (p<0.01), and food uptake by almost twofold (p<0.01). Voracity seems to be MSG-dose-dependent and the increase was identical in both genders.

Glutamic acid (GLU) is the most common amino acid in animal protein, and accounts for some 16% of meat protein, and some 20% of milk protein weight. That is infants who daily consume up to 5 g/kg body weight of protein (Koletzko, Pädiat. Prax. (2002), 62:386-388), consume as much as 1 g/kg body weight of GLU. GLU is also the physiological ligand of the taste receptor umami, the dominant taste of food containing L-GLU, like chicken broth, meat extracts, ageing cheese. Umami is responsible for the immediate sensory effect of monosodium glutamate (MSG) on the palatability of food. MSG is used as flavouring agent.

But it has long been known that MSG can also intoxicate arcuate nucleus neurones.

In 1969, Olney and co-workers reported on brain lesions, obesity, and other disturbances in mice (Olney, Science (1969) 164:719-21), and in an infant rhesus monkey (Olney, Science (1969), 166:386-8) treated with MSG. In 1976, Holzwarth-McBride and co-workers (Holzwarth-McBride, Anat. Rec. (1976), 197-205) investigated the effect of the MSG induced lesion of the arcuate nucleus by measuring catecholamine content in this nucleus and the median eminence of the mouse hypothalamus. The two major characteristics of MSG-induced arcuate nucleus damage hitherto described, are voracity, and impaired growth hormone (GH) secretion. However, all of these studies focussed on parenterally administered MSG. We demonstrated that MSG maintains its toxicity even when administered orally.

These findings are alarming, and throw doubts upon the unscrupulousness of current use of the flavouring agent MSG. L-Glutamic acid was evaluated by the Joint FAO/WHO Expert Committee on Food Additives (JECFA) in 1988. The JECFA allocated an “acceptable daily intake not specified” to glutamic acid and its salts with no additional risk to infants. The Scientific Committee for Food (SCF) of the European Commission reached a similar evaluation in 1991. The conclusions of a subsequent review by the Federation of American Societies for Experimental Biology (FASEB) and the Federal Drug Administration concurred with the safety evaluation of JECFA and the SCF (Walker, J. Nutr. (2000), 130:149 S-52S). MSG can be added at concentrations of up to 10 g per kg food (European Parliament and Council Directive 95/2/EC). Ca. 3 g MSG are added per kg potato chips (Greiff, Bahlsen-Lorenz company, personal communication, 2002), ca. 3-6 g are added per kg meat products.

We used a medium (2.5 g per day per adult animal) and a high (5 g per day per adult animal) MSG diet, accounting for some 10%, respectively some 20% of the daily amount of food. Yet, rat chow is dried food. Assuming a water content of some 70% in an ordinary breakfast sausage, 6 g MSG per kg meat product equals some 2% MSG in the dry product. I.e., the medium concentrations of GLU used in our animals, surpassed the concentration that is currently added to modern industrial food, by only the factor five.

The present study demonstrates that a widely used nutritional mono-substance—the flavouring agent monosodium glutamate—at concentrations that only slightly surpass those found in everyday human food, exhibits significant potential for damaging the hypothalamic regulation of appetite. Though the experimental part of this study was performed in rodents, and though it remains to be elucidated whether rodents are more sensitive to MSG than humans, uneasiness remains when considering that world-wide MSG production has increased from 200,000 (1969), to 270,000 (1979), to 800,000 (2001), and to 1,500,000 tons/year in 2004 (Schmid (2002) Taschenatlas der Biotechnologie und Gentechnik, Weinheim, Wiley-VCH, and personal communication, 2005).

Obesity results in particular from a nutritional imbalance. In view of the present findings, it may be considered whether not voracity is the disease that needs to be addressed in the first place. It has been shown that obesity associates with growth hormone (GH) secretory dysfunction. Twenty-four hour integrated concentrations of GH were lower in young, obese subjects than in young subjects who were lean (Meistas, Metabolsim (1982), 31:1224-8). Veldhuis et al. 1991 examined the mechanisms underlying the reduced circulating GH concentrations in obese subjects. Obese men had fewer GH secretory bursts, and both GH secretion rate and GH burst frequency were negatively correlated with the degree of obesity (Veldhuis, J. Clin. Endocrinol. Metab. (1991) 72:51-9). But since obesity results from a nutritional imbalance, i.e., obesity results from voracity—it is envisaged that both the damage in the regulation of appetite and the impaired growth hormone secretion result from world-wide supraphysiological GLU consumption.

### Example 4

### Medical Intervention of Human Obesity with Memantine

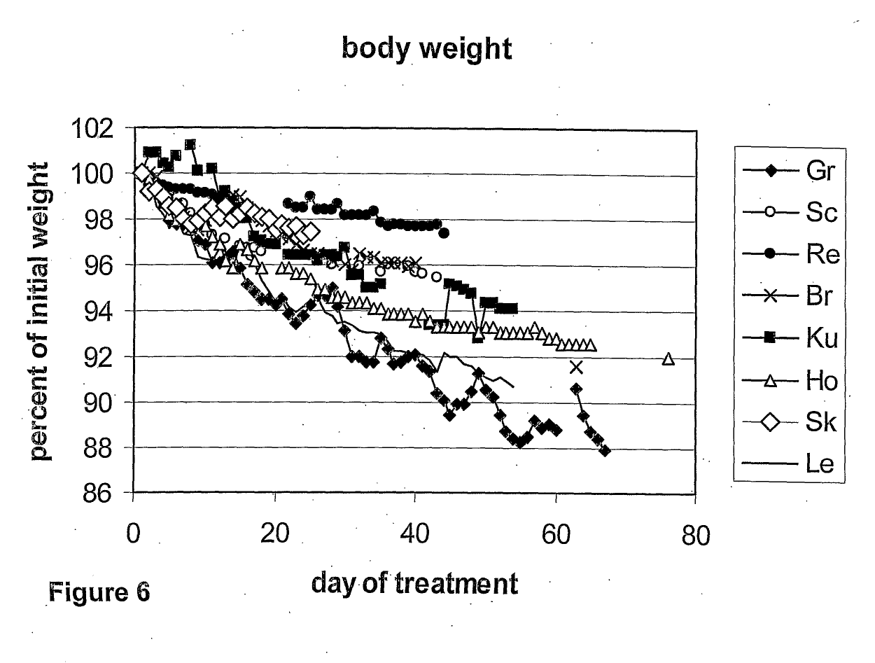

The following preliminary and limited clinical trials have been carried out in the private practice of the inventor. The trials with memantine are carried out since Feb. 2, 2005, in eight obese, but otherwise healthy patients, 6 females, and 2 males (Table 1; FIG. 6).

TABLE 1

| | | | Weight | | | |
|---|---|---|---|---|---|---|
| | | | (kg) | BMI | Weight | |
| | | Age | before | before | (kg) after | Days of |
| | | (y) | treatment | treatment | treatment | treatment |
| Gr | female | 34 | 126.6 | 43.3 | 111.3 | 67 |
| Sc | female | 31 | 90.6 | 33.3 | 86.6 | 49 |
| Re | female | 33 | 93.5 | 32.6 | 91.3 | 56 |
| Br | female | 34 | 122.3 | 42.8 | 117.5 | 40 |
| Ku | female | 23 | 111.0 | 38.6 | 104.4 | 56 |
| Ho | female | 62 | 77.6 | 27.5 | 71.4 | 77 |
| Sk | male | 36 | 148.6 | 38.3 | 144.8 | 25 |
| Le | male | 17 | 109.6 | 31.7 | 99.4 | 54 |

The patients reported on significant decrease in appetite, in particular, they reported that the usual late afternoon or evening binge-eating disorders had complete disappeared, already at the second day of treatment. Apart from short moments of mild dizziness at the first day, there were no side effects during the initial treatment period.

These data clearly document the efficient use of Memantine in the medical intervention of obesity.

1. (canceled)

2. A method for the prevention, amelioration and/or treatment of disorders of metabolism influencing body weight, an eating disorder and/or in the regulation of appetite comprising the step of administering a therapeutically effective amount of an N-methyl D-aspartate receptor antagonist (NMDA receptor antagonist) or a pharmaceutically acceptable salt or prodrug thereof.

3. The method of claim 2, wherein said NMDA receptor antagonist is selected from the group consisting of memantine or a pharmaceutically acceptable salt or a prodrug thereof, neramexane or a pharmaceutically acceptable salt or a prodrug thereof, MRZ 2/579 or a pharmaceutically acceptable salt or a prodrug thereof, selfotel or a pharmaceutically acceptable salt or a prodrug thereof, aptiganel or a pharmaceutically acceptable salt or a prodrug thereof, licostinel or a pharmaceutically acceptable salt or a prodrug thereof, ACEA 1021 or a pharmaceutically acceptable salt or a prodrug thereof, gavestinel or a pharmaceutically acceptable salt or a prodrug thereof, eliprodil or a pharmaceutically acceptable salt or a prodrug thereof, CP-101606 or a pharmaceutically acceptable salt or a prodrug thereof, and Co101244 or a pharmaceutically acceptable salt or a prodrug thereof.

4. The method of claim 3, wherein said NMDA receptor antagonist is selected from the group consisting of memantine or a pharmaceutically acceptable salt or a prodrug thereof, neramexane or a pharmaceutically acceptable salt or a prodrug thereof, MRZ 2/579 or a pharmaceutically acceptable salt or a prodrug thereof, ACEA 1021 or a pharmaceutically acceptable salt or a prodrug thereof, CP-101606 or a pharmaceutically acceptable salt or a prodrug thereof, Co101244 or a pharmaceutically acceptable salt or a prodrug thereof and eliprodil or a pharmaceutically acceptable salt or a prodrug thereof.

5. The method of claim 4, wherein said NMDA receptor antagonist is memantine or a pharmaceutically acceptable salt or a prodrug thereof.

6. The method of claim 4, wherein said NMDA receptor antagonist is neramexane or a pharmaceutically acceptable salt or prodrug thereof.

7. The method of claim 2, wherein said pharmaceutically acceptable salt is hydrochloride.

8. The method of claim 2, wherein said pharmaceutically acceptable salt is mesylate.

9. The method of claim 2, wherein said disorder of metabolism is a disease related to or involving higher levels of triglycerides.

10. The method of claim 2, wherein said disorder of metabolism is obesity.

11. The method of claim 10, whereby said obesity is overweight.

12. The method of claim 11, wherein said overweight is defined as a body mass index (BMI) between 25 to 30 kg/m² of the subject to be treated.

13. The method of claim 10, wherein said obesity is adipositas.

14. The method of claim 13, wherein said adipositas is defined as a body mass index (BMI) of higher than 30 kg/m² of the subject to be treated.

15. The method of claim 10, wherein said obesity is characterized as 20% or more extra body fat in the subject to be treated.

16. The method of claim 2, wherein said regulation of appetite is the suppression and/or depression of appetite.

17. A method for the prevention of secondary disorders of a disorder related to obesity or a secondary disorder related to increased body weight comprising the step of administering a therapeutically effective amount of an N-methyl D-aspartate receptor antagonist (NMDA receptor antagonist) or a pharmaceutically acceptable salt or prodrug thereof.

18. The method of claim 17, wherein said secondary disorder is selected from the group consisting of diabetes type 2, high blood pressure (hypertension), cardiovascular diseases, cancer, problems with sexual function and disorder of the muscular or bone system.
